(19) United States
(12) Patent Application Publication
(10) Pub. No.: US 2010/0152608 A1
(43) Pub. Date: Jun. 17, 2010

(54) CHRONICALLY IMPLANTED ABDOMINAL PRESSURE SENSOR FOR CONTINUOUS AMBULATORY ASSESSMENT OF RENAL FUNCTIONS

(76) Inventors: John D. HATLESTAD, Maplewood, MN (US); Binh C. TRAN, Minneapolis, MN (US); Bin MI, Plymouth, MN (US); Ronald W. HEIL, JR., Roseville, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/537,403

(22) Filed: Aug. 7, 2009

Related U.S. Application Data
(60) Provisional application No. 61/096,364, filed on Sep. 12, 2008.

(51) Int. Cl. A61B 5/00; A61B 5/11

(52) U.S. Cl. 600/561; 600/595

(57) ABSTRACT

An implantable system for ambulatory monitoring of a high-risk heart failure patient includes a first pressure sensor implantable within an abdomen of the patient for sensing and generating an output representative of a baseline intra-abdominal pressure value of the patient and for chronically sensing and generating an output representative of an intra-abdominal pressure value of the patient at periodic intervals. At least one second implantable sensor is provided for sensing and generating an output representative of a second physiological parameter of the patient. Additionally, the system includes a processor for correlating the output of the first pressure sensor and the second physiologic sensor, and for comparing differences between the baseline intra-abdominal pressure value and subsequent intra-abdominal pressure values. The processor can reside in another implantable device or in an external device/system.

### CROSS-REFERENCE TO RELATED APPLICATION

This application claims the benefit under 35 U.S.C §119 of U.S. Provisional Application No. 61/096,364, filed on Sep. 12, 2008, entitled “Chronically Implanted Abdominal Pressure Sensor for Continuous Ambulatory Assessment of Renal Functions,” which is herein incorporated by reference in its entirety.

### TECHNICAL FIELD

The present invention relates to the field of monitoring high-risk heart failure patients. In particular, the present invention relates to chronically measuring an intra-abdominal pressure to monitor renal functions related to heart failure.

### BACKGROUND

Renal functions are important co-morbidities/symptoms for acute decompensated heart failure (ADHF). In many cases, renal failure and dysfunction coexist with cases of heart failure. One method of monitoring renal functions is to measure the intra-abdominal pressure of the patient. High intra-abdominal pressures include intra-abdominal hypertension (IAH), generally accepted as an intra-abdominal pressure greater than about 12-25 mmHg. Intra-abdominal hypertension is associated with various ailments, such as ascites, renal problems and heart failure decompensation. For example, for a person with normal blood pressure and an elevated intra-abdominal pressure, the intra-abdominal pressure may cause collapse of the capillaries and veins, compromising perfusion to the visceral organs, in particular, the kidneys. There is a continuing need for improved and minimally invasive methods of measuring intra-abdominal pressure.

### SUMMARY

In a first aspect, the present invention is a method of monitoring heart functions of a patient. The method includes measuring a first intra-abdominal pressure value of the patient using a first pressure sensor chronically implanted at least partially within an abdomen of the patient, chronically measuring a plurality of second intra-abdominal pressure values of the patient at periodic intervals when the patient is ambulatory using the first pressure sensor, remotely transmitting the first and second intra-abdominal pressure values to one or both of an external device and an implant within the patient, associating the second intra-abdominal pressure values with an output from at least one second implantable physiologic sensor to obtain a plurality of correlated second intra-abdominal pressure values, comparing the first intra-abdominal pressure value to the associated second intra-abdominal pressure values, and associating a difference between the first intra-abdominal pressure value and any one or a plurality of the associated second intra-abdominal pressure values with a renal condition linked to heart function.

In a second aspect, the present invention is a method of monitoring heart functions of a patient. The method includes measuring a plurality of intra-abdominal pressure values of the patient when the patient is ambulatory using a first implanted pressure sensor and associating one or more elevated intra-abdominal pressure values with one or more signs of deteriorating heart condition.

In a third aspect, the present invention is a system for ambulatory monitoring of a high-risk heart failure patient. The system includes a first pressure sensor implantable within an abdomen of the patient for sensing and generating an output representative of a baseline intra-abdominal pressure value of the patient and for chronically sensing and generating an output representative of an intra-abdominal pressure value of the patient at periodic intervals. At least one second implantable sensor is provided for sensing and generating an output representative of a second physiological parameter of the patient. Additionally, the system includes a processor for correlating the output of the first pressure sensor and the second physiologic sensor, and for comparing differences between the baseline intra-abdominal pressure value and subsequent intra-abdominal pressure values. The processor can reside in another implantable device or in an external device/system.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram of a heart monitoring system according to an embodiment of the present invention.

FIG. 2 is a schematic diagram of a heart monitoring system according to an alternative embodiment of the present invention.

FIG. 3 is a schematic diagram of a method of monitoring high-risk heart failure patients according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

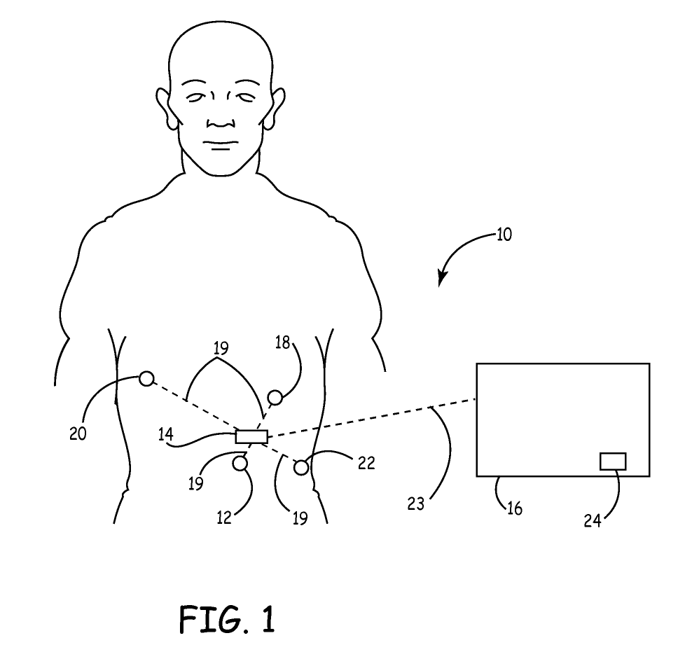

FIG. 1 shows a schematic diagram of a heart monitoring system 10 for predicting the early onset of heart failure, according to an embodiment of the present invention. As shown in FIG. 1, the heart monitoring system 10 includes a first pressure sensor 12, an implantable medical device (“IMD”) 14, and an external device/system 16. In the illustrated embodiment, the first pressure sensor 12 and the IMD 14 are both implanted within the patient for facilitating chronic ambulatory assessment of a patient's renal functions and/or other physiological conditions, e.g., ascites. As shown, the first pressure sensor 12 is implanted within the patient's abdomen, with the IMD 14 implanted subcutaneously at a location remote from the first pressure sensor 12. As further shown, the external device/system 16 is located external to the patient. As discussed in detail below, in various embodiments, the first pressure sensor 12 and the IMD 14 are in communication with one another, and one or both of the foregoing is configured to be in communication with the external device/system 16.

As explained in detail below, according to the various embodiments of the present invention, the heart monitoring system 10 senses and measures intra-abdominal pressures, and utilizes this pressure data to assess patient renal functions, which, along with parameters such as fluid volume, impedance, and posture, are correlated to various symptoms and co-morbidities of heart failure. Measuring the intra-abdominal pressure of a patient enables the heart monitoring system 10 to measure and monitor heart-related diagnostics, such as renal function and fluid status. In some embodiments, the heart monitoring system 10 can provide an indicator of the autonomic balance, fluid status of a patient and orthopnea. By measuring these diagnostics, the heart monitoring system 10 provides a minimally invasive system for predicting the early onset of heart failure.

The first pressure sensor 12 chronically measures the intra-abdominal pressure in ambulatory patients. In one embodiment, the first pressure sensor 12 is implanted within the abdominal wall, which provides a firm structure to which the first pressure sensor 12 is attached. In addition, attaching the first pressure sensor 12 to the abdominal wall provides easy access to the implantation site, allowing for the first pressure sensor 12 to be implanted in a minimally invasive manner. For example, the first pressure sensor 12 may be introduced into the abdomen through the patient's navel, which is commonly used for laparoscopic procedures. In addition, by anchoring the first pressure sensor 12 to the abdominal wall, there is a clear acoustical path to the IMD 14 such that wireless acoustic communication between the first pressure sensor 12 and the IMD 14 is possible. Of course, in various other embodiments, the first pressure sensor 12 is anchored within the abdomen but not within the abdominal wall.

The first pressure sensor 12 is implantable substantially anywhere within the abdomen and is located based on the condition to be monitored. For example, when it is desired for the heart monitoring system to monitor primarily renal function, it may be advantageous to implant the first pressure sensor 12 near the kidneys. When it is desired for the heart monitoring system to monitor ascites, it may be advantageous to implant the first pressure sensor 12 near the groin. The first pressure sensor 12 may also be anchored to an organ, such as the liver or spleen, or within one of several vessels in the abdomen. Any anchoring means known in the art and still others yet to be developed may be used to anchor the first pressure sensor 12 within the abdomen without departing from the scope of the present invention.

In the illustrated embodiment, the heart monitoring system 10 also includes an optional second pressure sensor 18 implantable within the abdomen of the patient for measuring the intra-abdominal pressure of the patient. While the second pressure sensor 18 functions in the same manner as the first pressure sensor 12 and is implanted and anchored within the abdomen in the same manner as the first pressure sensor 12, the second pressure sensor 18 is spaced from the first pressure sensor 12 in order to obtain a reading of the intra-abdominal pressure from a different location within the abdomen. When the heart monitoring system 10 includes both the first pressure sensor 12 and the second pressure sensor 18, the signals from both of the pressure sensors 12, 18 are sent to the IMD 14 which averages and correlates the pressures measured by the first pressure sensor 12 and the second pressure sensor 18. In this manner, a more accurate intra-abdominal pressure measurement may be determined. While the heart monitoring system 10 is discussed as including two pressure sensors 12, 18, the heart monitoring system 10 may include only a single pressure sensor or, alternatively, may include more than two pressure sensors located at various locations within the abdomen without departing from the intended scope of the present invention.

The pressure sensors 12, 18 can have any structure and configuration providing the desired functionality. As will be appreciated, the pressure sensors 12, 18 are configured to sense the intra-abdominal pressure, and transmit the pressure data to the IMD 14 and/or the external device/system 16. In various embodiments, the pressure sensors 12, 18 each include one or more pressure sensitive transducers configured to produce a signal (i.e. current or voltage) indicative of pressure within the patient's abdomen. In various embodiments, the pressure transducer can be a micro-electrical-mechanical system (MEMS) device, which as will be appreciated, utilizes semiconductor techniques to build microscopic mechanical structures in a substrate made from silicon or similar materials. In various embodiments, the pressure transducer can include a micro-machined capacitive or piezoresistive transducer. Other pressure transducer technologies, such as resistive strain gages and piezoelectric devices, are known in the art and can also be employed in the pressure sensors 12, 18.

As will be appreciated, the pressure sensors 12, 18 include various circuitry and other components to facilitate the functional aspects of the sensors. For example, the pressure sensors 12, 18 include telemetry components to facilitate communication between the sensors 12, 18 and the IMD 14 and/or the external device/system 16. In various embodiments, the pressure sensor 12 and/or the pressure sensor 18 may include a battery to supply operating power for the sensor components. In various other embodiments, the pressure sensor 12 and/or the pressure sensor 18 may be powered from an external source. For example, in one embodiment, the pressure sensor 12 and/or 18 includes an acoustic transducer configured to generate an output voltage in response to acoustic energy received from another device (e.g., the IMD 14 and/or the external device/system 16). As will be appreciated, the transducer output may then be utilized to charge one or more capacitors, which are discharged to provide operating power for the other active components of the sensor. Various suitable systems and techniques for recharging or acoustically powering another implant are disclosed in, for example, commonly assigned U.S. Pat. No. 6,432,050 to Porat et al. and U.S. Pat. No. 7,024,248 to Penner et al., the disclosures of which are incorporated herein by reference in their entireties. In various other embodiments, the sensor 12 and/or the sensor 18 may be powered by an alternative energy source, e.g., inductive or radio-frequency electromagnetic energy.

The IMD 14 can be any implantable medical device, whether now known or later developed, configured for providing the desired functionality as discussed herein. In various embodiments, the IMD 14 includes internal processing components, e.g., a processor such as a central processing unit or integrated circuit, for processing signals from the first and second pressure sensors 12, 18, as well as other sensors implanted in the patient as explained below. Additionally, as will be appreciated, the IMD 14 includes, in various embodiments, memory, a power supply, telemetry systems, and/or other functional components necessary to provide the desired functionality. In one embodiment, the IMD 14 is a cardiac rhythm management (“CRM”) device such as a pacemaker, an implantable cardioverting defibrillator (“ICD”), or cardiac resynchronization therapy (“CRT”) device (which may or may not include defibrillation capabilities). In general, pacemakers, ICDs, and CRT devices are well known in the art, and need not be described in further detail herein.

Of course, the IMD 14 need not be a CRM device. In one embodiment, for example, the IMD 14 is a drug delivery system. In various embodiments, the IMD 14 is a monitoring device only, e.g., it does not itself provide any therapeutic stimuli.

In one embodiment, the IMD 14 operates as an implantable monitor/repeater device, and thus includes a processor that receives the pressure sensor 12, 18 output, and manipulates and processes the sensor output as described herein. For example, the processor in the IMD 14 could generate trends, waveforms, and the like, which are then transmitted to the external device/system 16. In one embodiment, the IMD 14 is operable as a data storage and transmission device, whereby the IMD 14 stores the raw pressure sensor output data and transmits it to the external device/system 16 as desired. In one embodiment, the IMD 14 is a CRM device that receives the output signal from the pressure sensors 12, 18, which represents the sensed intra-abdominal pressure data, and uses this pressure data in a closed-loop manner to adaptively adjust therapy parameters. The foregoing therapeutic functionality can be provided in addition to or in lieu of the monitoring and/or data storage functions discussed above.

In the illustrated embodiment, the IMD 14 is operatively coupled to the first and second pressure sensors 12, 18 via a communication link 19. The IMD 14 is configured to receive and process the output signal(s) from the pressure sensors 12, 18.

In one embodiment, the IMD 14 is configured to receive the output signal(s) from the pressure sensors 12, 18 and measure a baseline intra-abdominal pressure. In various embodiments, the baseline pressure measurement is determined by the first and/or second pressure sensors 12, 18 according to a pre-determined regimen (i.e., daily, hourly, semi-weekly, etc.) while the patient is positioned in one or more specified postures and/or while the patient is performing one or more specified activities. In one embodiment, the baseline pressure measurement is taken at least daily. In various embodiments, to enhance the accuracy of the baseline intra-abdominal pressure measurement, it is determined based on multiple or continuous readings sensed and recorded over a period of time. For example, in one embodiment, the intra-abdominal pressure measurement is taken substantially continuously for a pre-determined time period so as to generate a pressure data curve, which can then be processed in a variety of ways to determine an accurate representative baseline intra-abdominal pressure value. In another embodiment, multiple pressure measurements are performed at pre-determined sampling rates, and the results averaged to define the baseline intra-abdominal pressure value. Still other schemes for obtaining the baseline intra-abdominal pressure value will become apparent to those skilled in the art based on the foregoing.

The IMD 14 can also be configured to obtain atmospheric or ambient (i.e. barometric) pressure data from another source and calibrate or correct the measured intra-abdominal pressure values for changes in barometric pressure.

In addition, the IMD 14 may be programmed to account for factors that may affect the intra-abdominal pressure because the first pressure sensor 12 and the second pressure sensor 18 measure the intra-abdominal pressure of the patient chronically while the patient is ambulatory. External factors may effect the intra-abdominal pressure of the patient. For example, the intra-abdominal pressure reading of the patient may be effected by the position or posture of the patient. The intra-abdominal pressure reading may also be affected depending on whether the patient is engaged in an activity requiring the patient to expend energy.

To account for the posture or activity of the patient, the heart monitoring system 10 may optionally include a posture sensor 20 or an activity sensor 22 for determining the posture of the patient or if the patient is performing an activity while the intra-abdominal pressure of the patient is being measured. Various implantable posture and activity sensors, e.g., those utilizing accelerometers as the active elements, are known in the art, and thus the specific configurations of the sensors 20, 22 are not discussed in detail here. Examples of posture detection systems are disclosed in, for example, commonly assigned U.S. Patent Application 2007/0118056 to Wang et al. and U.S. Patent Application 2007/0115277 to Wang et al., the disclosures of which are incorporated herein by reference in their entireties. In short, the posture and activity sensors 20, 22, can take on any form or configuration, whether now known or later developed. Additionally, as will be appreciated, in various embodiments, the posture and/or activity sensors 20, 22 can be located within the enclosure of the IMD 14.

The posture or activity sensors 20, 22 may gather a set of initial readings under known conditions in order to formulate a baseline to compare subsequent readings. In one embodiment, the initial readings are taken when the patient is in a supine resting position. The pressure, posture and activity measurements are transmitted to the IMD 14 (or the external device/system 16) to be recorded and trended. The measurements and trends can then be retrieved and viewed by the clinician.

The IMD 14 trends the data by comparing the differences between a baseline intra-abdominal pressure and subsequent intra-abdominal pressures along with other factors, including time of day, posture of the patient during the measurement and activity of the patient during the measurement. In various embodiments, the IMD 14 can, itself, determine an optimal therapy to treat heart failure or other condition based on the trends of the intra-abdominal pressure readings and the differences between the baseline intra-abdominal pressure and subsequent intra-abdominal pressures.

The IMD 14, the pressure sensors 12, 18, the posture sensor 20 and the activity sensor 22 can be in communication with each other using such means such as acoustic, RF, or optical methods, for example. Various suitable systems and techniques for intra-body acoustic communication between the IMD 14 and the sensors 12, 18, 20, 22 are disclosed in, for example, commonly assigned U.S. Patent Application Publication 2006/0009818 to Von Arx, et al., and U.S. Pat. No. 7,024,248 to Penner et al., the disclosures of which are incorporated herein by reference in their entireties. Alternatively, the IMD 14 can have a wired connection to the pressure sensors 12, 18 and the posture sensor 20 and the activity sensor 22.

In the illustrated embodiment, the external device/system 16 is in communication with the IMD 14 and/or any of the sensors 12, 18, 20, 22 via a communication link 23. In various embodiments, the external device/system operates to allow a patient, a physician or other caregiver to communicate with the IMD 14 and/or any of the sensors 12, 18, 20, 22. In various embodiments, the external device/system 16 can, itself, determine an optimal therapy to treat heart failure or other condition based on the trends of the intra-abdominal pressure readings and the differences between the baseline intra-abdominal pressure and subsequent intra-abdominal pressures.

The specific form and functionality of the external device/system 16 is not limited to any particular type of device or system. In one embodiment, the external device/system 16 includes an external programmer, monitor, or combinations thereof. For example, in one embodiment, the external device/system 16 is an external, hand-held programmer utilized by the physician or other caregiver to access data, including intra-abdominal pressure data, stored in memory within the IMD 14, as well as to program operating parameters for the IMD 14 and/or any of the sensors 12, 18, 20, 22. In one embodiment, the external device/system 16 is a local monitor, e.g., a bedside monitor located in the patient's residence, configured to, among other things, retrieve intra-abdominal pressure data from the IMD 14 and store such data in its own memory or transmit the data to another device or system. In one embodiment, the external device/system 16 is a wearable monitor that is worn and carried by the patient and in direct communication with sensors 12, 18, which may be particularly advantageous for providing ambulatory intra-abdominal pressure data. In one embodiment the external device can be an external reader such as a handheld reader or a dialysis counsel.

In various embodiments, the external device/system 16 includes a remote patient monitoring and management system including an external device (e.g., a local monitor or repeater) coupled to the IMD 14 via the communication link 23, a network coupled to the external device, and a remote device/system coupled to the network. Such a patient management system allows a physician or other caregiver to communicate with the IMD 14 and/or the sensors 12, 18, 20, 22 through the remote device in a distant location (e.g., at the physician's office while the patient is at home). As will be appreciated, the patient management system may include additional memory, e.g., a patient database and processing capabilities. One exemplary remote patient management system that can be incorporated into the external device/system 16 is the LATITUDE patient management system available from Boston Scientific Corporation.

Communication between the IMD 14 and the external device/system 16 can be accomplished through any suitable communication means. In one embodiment, the communication link 23 is an acoustic link to facilitate acoustic telemetry between the IMD 14 and the external device/system 16. Various suitable systems and techniques for communication between an implantable device such as the IMD 14 and an external device or system are disclosed in, for example, commonly assigned U.S. Patent Application Publication 2006/0009818 to Von Arx, et al., and U.S. Pat. No. 7,024,248 to Penner et al., the disclosures of which are incorporated herein by reference in their entireties. In one embodiment, the communication link 23 is an inductive telemetry link. In one embodiment, the communication link 23 is a far-field radio-frequency telemetry link. Still other communication methods/schemes for providing data transmission and other communication between the external device/system 16 and the IMD 14 will be apparent to those skilled in the art based on the foregoing.

In various embodiments, the external device/system 16 includes a display, alarm capabilities, or other means for communicating the retrieved intra-abdominal pressure data to the patient, the physician or other caregiver, or all of the above. In the illustrated embodiment, for example, the external device/system 16 includes an indicator 24 accessible by the patient and/or physician. In one embodiment, the indicator 24 activates when the intra-abdominal pressure reaches a predetermined value. In another embodiment, the indicator 24 activates when a difference between the baseline intra-abdominal pressure and subsequent intra-abdominal pressures is greater than a predetermined value. In one embodiment, the predetermined value is about 8 mmHg, about 12 mmHg, or about 16 mmHg. The indicator 24 can be designed to notify the patient, a clinician or a physician of significant intra-abdominal pressure elevation. If the indicator 24 is designed to notify the patient, for example through a wristwatch remotely connected to first pressure sensor 12, the patient is alerted that he/she should either schedule a visit with a physician, report to an emergency room or take other preventative measures. Because the external device/system 16 is remotely connected to the IMD 14, a clinician or physician does not have to be in close proximity to the patient to be notified that the intra-abdominal pressure of the patient has elevated to a potentially dangerous level.

Alternatively, the indicator 24 may be programmed to activate only if the difference in the baseline intra-abdominal pressure and subsequent intra-abdominal pressure remains greater than the predetermined value for more than one or multiple readings. The external device/system 16 may also be programmed to increase the number of readings taken within a certain period of time if the difference in the baseline intra-abdominal pressure and the subsequent intra-abdominal pressure is greater than the predetermined level. Although the indicator 24 is discussed as activating when the difference between the baseline intra-abdominal pressure and subsequent intra-abdominal pressure is greater than a predetermined value, other factors may also be used to determine when the indicator 24 is activated without departing from the intended scope of the present invention.

The trends in intra-abdominal pressure can also be used to understand a patient's compliance and adherence to a specified diet or medication. Patients with elevated intra-abdominal pressure are typically on a regimented diet that recommend against or prohibit ingestion of particular types of foods that may increase the intra-abdominal pressure. By examining the trends of the intra-abdominal pressure, the clinician may be able to determine if a patient deviated from the diet or neglected to take his/her medication if the intra-abdominal pressure of the patient increased by an atypical amount.

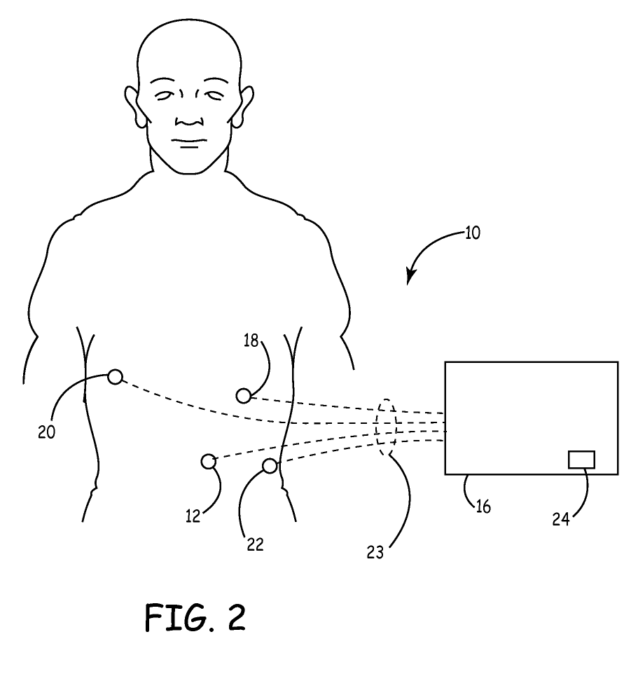

FIG. 2 is a schematic diagram of the heart monitoring system 10 according to an alternative embodiment of the present invention. As shown, in the embodiment of FIG. 2, the heart monitoring system 10 includes a plurality of pressure sensors 12, 18, and an external device/system 16. As further shown, the system 10 of FIG. 2 includes additional implantable sensors, including a posture sensor 20 and an activity sensor 22. The sensors 12, 18, 20, and 22 are communicably coupled to the external device/system 16 via communication link 23. Each of the sensors 12, 18, 20, and 22, and the external device/system 16 can be configured in substantially the same or an identical manner to the corresponding elements of the system 10 of FIG. 1, and thus can provide the same ranges of functionality. In the embodiment of FIG. 2, however, the sensors do not communicate with another implanted device, i.e., the IMD 14 of the embodiment of FIG. 1. Thus, in the embodiment of FIG. 2, the outputs from the sensors 12, 18, 20, and 22 are transmitted directly to the external device/system 16 via the communication link 23. This communication can be accomplished via any suitable means or technique, whether now known or later developed.

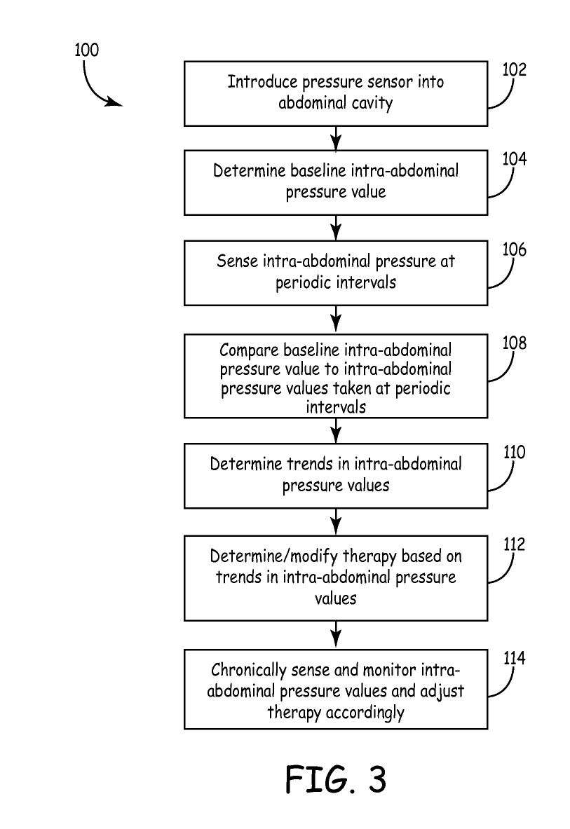

FIG. 3 is a schematic diagram of a method 100 of monitoring high-risk heart failure patients according to an embodiment of the present invention. The method 100 includes first introducing a first pressure sensor 12 into an abdomen of the patient, (Block 102). In one embodiment, the first pressure sensor 12 is attached to an abdominal wall of the patient. Optionally, an additional pressure sensor 18, a posture sensor 20 and/or an activity sensor 22 may also be implanted in the patient's body. A set of initial readings of the intra-abdominal pressure are taken with the sensors 12, 18, 20, 22 to determine a baseline pressure in the abdomen, (Block 104). In one embodiment, the baseline pressure is determined based on pressure readings taken while the patient is ambulatory. The readings are optionally also taken when the patient is positioned in a particular posture or performing a particular activity.

Once the baseline intra-abdominal pressure is determined, the pressure in the abdomen is sensed at periodic intervals and communicated to a processor, which could, in various embodiments, be located within the IMD 14 and/or the external device/system 16, (Block 106). The intra-abdominal pressures are recorded and trended at each periodic interval and compared to the baseline pressure, (Block 108). In one embodiment, the subsequent intra-abdominal pressures are sensed or measured while the patient is ambulatory.

The trends are based on the differences between the baseline intra-abdominal pressure and the subsequent intra-abdominal pressures taken, (Block 110). The trends may be used to determine therapy goals and profiles. A therapy may then be set based on the trends, (Block 112). Alternatively, or additionally, a clinician may be automatically notified by the external device/system 16 if predetermined conditions are detected. For example, in various embodiments, a rise in the difference between the baseline intra-abdominal pressure value and subsequent intra-abdominal pressure values can indicate the onset of ascites, renal problems, and/or early decompensation in heart failure patients. Thus, in various embodiments, the external device/system 16 and/or the IMD 14 are configured to automatically detect the occurrence of a rise in the intra-abdominal pressure relative to the baseline exceeding a predetermined threshold amount, so as to operate as a detection system for detecting conditions such as ascites, renal failure, and/or early decompensation.

For example, in various embodiments, the external device/system 16 includes a remote patient management system which in turn includes a detection and alert mechanism for notifying a clinician of significant abdominal pressure increases. In one embodiment, the pressure sensor 12, 18 and other implanted sensors, when present, are incorporated into an early decompensation detection system which is configured to generate physician alerts upon the occurrence of predetermined conditions, e.g., changes in intra-abdominal pressures exceeding a predetermined threshold amount. Exemplary automatic detection systems that can be employed in the present invention are disclosed in commonly assigned U.S. Patent Application Publication 2007/0213599 to Siejko, et. al., the disclosure of which is incorporated herein by reference in its entirety.

Even after the therapy has been determined and administered, the heart monitoring system 10 can optionally continue to take intra-abdominal pressure readings of the patient, (Block 114) The subsequent readings may be useful to determine whether the treatment is effective or whether the patient is following proper protocol.

The heart monitoring system of the present invention measures the intra-abdominal pressure of a patient by percutaneously deploying a pressure sensor into the abdomen of the patient. The process is minimally invasive and may be an outpatient procedure. In addition, because the pressure sensor is percutaneously introduced into the body, the risks of infection, bleeding, subclavian stenosis, etc. decreases. The pressures are communicated to a processor which determines trends between a baseline intra-abdominal pressure and subsequent intra-abdominal pressures. The trends in the intra-abdominal pressure of the patient are related to renal functions, which correspond to various symptoms and co-morbidities of heart failure. Thus, by measuring and studying the trends of the intra-abdominal pressure of a patient, early signs of worsening heart failure may be able to be diagnosed.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A method of monitoring heart functions of a patient, the method comprising:
measuring a first intra-abdominal pressure value of the patient using a first pressure sensor chronically implanted at least partially within an abdomen of the patient;
chronically measuring a plurality of second intra-abdominal pressure values of the patient at periodic intervals when the patient is ambulatory using the first pressure sensor;
remotely transmitting the first and second intra-abdominal pressure values to one or both of an external device and an implant within the patient;
associating the second intra-abdominal pressure values with an output from at least one second implantable physiologic sensor to obtain a plurality of correlated second intra-abdominal pressure values;
comparing the first intra-abdominal pressure value to the associated second intra-abdominal pressure values; and
associating a difference between the first intra-abdominal pressure value and any one or a plurality of the associated second intra-abdominal pressure values with a renal condition linked to heart function.

2. The method of claim 1, further comprising adjusting a therapy based on a difference between the first intra-abdominal pressure value and any one or a plurality of the associated second intra-abdominal pressure values.

3. The method of claim 2, wherein the therapy is a renal therapy.

4. The method of claim 1, further comprising sending a signal to an external device if the difference between the first intra-abdominal pressure and any one or a plurality of the associated second intra-abdominal pressure values is greater than a predetermined value.

5. The method of claim 4, wherein the predetermined value is about 8 mmHg.

6. The method of claim 1, wherein associating the second intra-abdominal pressure values with an output from at least one second implantable physiologic sensor includes associating the second intra-abdominal pressure values with an output from an implanted posture sensor and/or an output from an implanted activity sensor.

7. The method of claim 1, wherein measuring the first intra-abdominal pressure values further includes sensing the intra-abdominal pressure using a second pressure sensor implanted at least partially within the abdomen of the patient, and averaging an output of the first and second pressure sensors to obtain the first intra-abdominal pressure value.

8. The method of claim 7, wherein measuring the plurality of second intra-abdominal pressure values includes sensing intra-abdominal pressure at the periodic intervals using the second pressure sensor, and averaging an output of the first and second pressure sensors generated at the periodic intervals.

9. The method of claim 1, wherein associating the difference between the first intra-abdominal pressure value and any one or a plurality of the second intra-abdominal pressure values includes detecting an onset of renal failure, ascites, and/or early decompensation.

10. The method of claim 9, further comprising alerting a clinician upon detection of the onset of renal failure, ascites, and/or early decompensation.

11. The method of claim 1, wherein remotely transmitting the first and second intra-abdominal pressure values to one or both of an external device and an implant within the patient includes remotely transmitting the values through acoustic, RF, or optical communication.

12. A method of monitoring heart functions of a patient, the method comprising:
measuring a plurality of intra-abdominal pressure values of the patient when the patient is ambulatory using a first implanted pressure sensor; and
associating one or more elevated intra-abdominal pressure values with one or more signs of deteriorating heart condition.

13. The method of claim 12, wherein the signs of deteriorating heart condition is one or more of ascites and compromised renal function.

14. The method of claim 12, further comprising remotely transmitting the intra-abdominal pressure values to one or both of an external device and an implant within the patient.

15. The method of claim 14, wherein remotely transmitting the intra-abdominal pressure values to one or both of an external device and an implant within the patient includes remotely transmitting the values through acoustic, RF, or optical communication.

16. The method of claim 12, wherein measuring a plurality of intra-abdominal pressure values includes measuring a first intra-abdominal pressure value of the patient as a baseline intra-abdominal pressure value and chronically measuring a plurality of second intra-abdominal pressure values of the patient at periodic intervals.

17. The method of claim 16, further comprising associating the first and second intra-abdominal pressure values with an output from at least one second implantable physiologic sensor to obtain a plurality of associated second intra-abdominal pressure values.

18. The method of claim 17, wherein associating one or more elevated intra-abdominal pressure values includes associating a difference between the first intra-abdominal pressure value and any one or a plurality of the associated second intra-abdominal pressure values to a renal condition linked to heart function.

19. The method of claim 12, wherein measuring the plurality of intra-abdominal pressure values further includes measuring the intra-abdominal pressure values using a second pressure sensor implanted at least partially within the abdomen of the patient, and averaging an output of the first and second pressure sensors to obtain the intra-abdominal pressure values.

20. An implantable system for ambulatory monitoring of a high-risk heart failure patient comprising:
a first pressure sensor implantable within an abdomen of the patient for sensing and generating an output representative of a baseline intra-abdominal pressure value of the patient and for chronically sensing and generating an output representative of an intra-abdominal pressure value of the patient at periodic intervals;
at least one second implantable sensor for sensing and generating an output representative of a second physiological parameter of the patient; and
a processor for correlating the output of the first pressure sensor and the output of the second physiologic sensor, and for comparing differences between the baseline intra-abdominal pressure value and subsequent intra-abdominal pressure values.

21. The implantable system of claim 20, further comprising an external system for determining a therapy based on the differences between the baseline intra-abdominal pressure value and subsequent intra-abdominal pressure values to treat heart failure.

22. The implantable system of claim 20, further comprising an external system for alerting a clinician when the difference between the baseline intra-abdominal pressure value and subsequent intra-abdominal pressure values exceeds a predetermined amount.

23. The implantable system of claim 20, wherein the first pressure sensor is configured to remotely transmit the output representative of the intra-abdominal pressure values to one or both of an external device and an implant in the patient.

24. The implantable system of claim 21, wherein the at least one second physiologic sensor is a posture sensor for determining posture of the patient.

25. The implantable system of claim 20, further comprising means for transmitting the baseline intra-abdominal pressure value and subsequent intra-abdominal pressure values to the processor.

26. The implantable system of claim 20, further comprising an indicator for alerting the patient when the difference between the baseline intra-abdominal pressure value and subsequent intra-abdominal pressure values is greater than a predetermined amount.

27. The implantable system of claim 20, further comprising an indicator for alerting a third party when the difference between the baseline intra-abdominal pressure. value and subsequent intra-abdominal pressure values is greater than a predetermined amount.

28. The implantable system of claim 20, further comprising a second pressure sensor implantable within the abdomen of the patient for measuring the baseline intra-abdominal pressure of the patient and for chronically measuring the intra-abdominal pressure of the patient, wherein the second pressure sensor is spaced from the first pressure sensor.

29. The implantable system of claim 28, wherein the processor is configured to average the intra-abdominal pressure values sensed by the first pressure sensor and the second pressure sensor.

30. The implantable system of claim 28, wherein the processor correlates the intra-abdominal pressure values sensed by the first pressure sensor and the second pressure sensor to the output of the second physiologic sensor.
